(19) United States
(12) Patent Application Publication
(10) Pub. No.: US 2009/0028944 A1
(43) Pub. Date: Jan. 29, 2009

(54) PHARMACEUTICAL COMPOSITIONS COMPRISING MESALAMINE

(76) Inventors: Balaji Sathurappan, Tirunelveli (IN); Anand Sankarnarayanan, Chennai (IN); Subhash Pandurang Gore, Solapur (IN); Narayanan Badri Vishwanathan, Chennai (IN); Indu Bhushan, Hyderabad (IN); Mailatur Sivaraman Mohan, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/173,875

(22) Filed: Jul. 16, 2008

Related U.S. Application Data
(60) Provisional application No. 60/949,903, filed on Jul. 16, 2007.

(51) Int. Cl. A61K 9/32; A61K 31/195; A61P 29/00; A61P 1/00; A61K 9/14

(52) U.S. Cl. 424/482; 514/567; 424/497

(57) ABSTRACT

Pharmaceutical compositions comprising mesalamine, wherein the compositions are free of a liphophilic matrix, and processes for preparing pharmaceutical compositions comprising mesalamine and being free of a liphophilic matrix.

The present invention relates to pharmaceutical compositions comprising mesalamine, including pharmaceutically acceptable salts, solvates, hydrates, and mixtures thereof, for oral administration, processes for preparing such compositions, and their methods of use.

Inflammatory bowel disease (IBD) refers to a group of chronic intestinal diseases characterized by inflammation of the bowel—the large or small intestine. The most common types of IBD are ulcerative colitis and Crohn's disease.

Mesalamine is an officially adopted name for the compound 5-amino-2-hydroxybenzoic acid, or 5-aminosalicylic acid (5-ASA), and is in a class of medications called anti-inflammatory agents. It is a crystalline powder that exhibits a needle-like morphology. Mesalamine has structural Formula I.

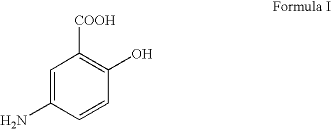

Mesalamine is slightly soluble in water, very slightly soluble in dehydrated alcohol, acetone, and methyl alcohol, insoluble in chloroform, ether, butyl alcohol, and ethyl acetate, and soluble in dilute hydrochloric acid and dilute alkali metal hydroxide solutions.

Mesalamine exhibits local action in the large intestine and has been formulated using various technologies to release the active at the site for the treatment of inflammation in the distal part of small intestine and colon and the treatment of inflammatory bowel disease (“IBD”).

Commercially, mesalamine is available in various dosage forms. For example, PENTASA™ (mesalamine 400 mg extended release capsules; Shire Inc., USA), ASACOL™ (mesalamine 200 mg and 400 mg delayed release tablets; Procter and Gamble, USA), and LIALDA™ (mesalamine 1.2 g delayed release tablets; Shire Inc., USA), for oral administration.

LIALDA™ tablets are coated with a gastro-resistant pH dependent polymer film, which breaks down at or above pH 7, normally in the terminal ileum where mesalamine then begins to be released from the tablet core. The tablet core contains mesalamine with hydrophilic and lipophilic excipients. LIALDA™ is indicated for the treatment of diseases such as mild to moderate ulcerative colitis.

U.S. Pat. Nos. 5,541,170 and 5,541,171 disclose delayed release tablets containing mesalamine. U.S. Pat. No. 5,686,105 describes enteric coated multi-particulate dosage forms for colonic delivery U.S. Pat. No. 6,773,720 discloses controlled release oral pharmaceutical compositions comprising mesalamine and a lipophilic matrix. U.S. Patent Application Publication No. 2003/0138495 discloses a method for preparation of mesalamine granules using extrusion technology. U.S. Patent Application Publication No. 2007/0059368 discloses modified release formulations of anti-irritability drugs. U.S. Patent Application Publication No. 2006/0210631 discloses a multi-particulate modified-release composition. U.S. Patent Application Publication No. 2006/0223787 discloses modified release formulations and a method of treating inflammatory bowel disease. International Application Publication No. WO 2004/087113 describes a delayed release single dosage unit composition of mesalamine for colonic delivery.

There remains a need for compositions providing ease of manufacturing and minimizing the use of pharmaceutical excipients, to deliver high unit doses of mesalamine.

### SUMMARY

The present invention relates to pharmaceutical compositions comprising mesalamine, including pharmaceutically acceptable salts, solvates, hydrates, enantiomers, polymorphs, and mixtures thereof, for oral administration, processes for preparing such compositions, and their methods of use.

Surprisingly, it has been observed that controlled release pharmaceutical compositions comprising mesalamine for oral administration, wherein said composition is free from a lipophilic matrix and provides a high unit dose of the active ingredient, exhibit desired in vitro release profiles and in vivo performance.

Thus a first aspect of the present invention provides pharmaceutical compositions comprising mesalamine or its salts, wherein said composition is free from a lipophilic matrix.

An aspect of the present invention provides orally administrable controlled release pharmaceutical unit dose compositions comprising more than about 0.9 g of mesalamine, optionally with pharmaceutically acceptable excipients, wherein said composition is free from a lipophilic matrix.

An aspect of the present invention provides controlled release pharmaceutical compositions comprising mesalamine particles, wherein the particles are granulated with a hydrophobic polymer and further embedded in a hydrophilic matrix.

An aspect of the present invention provides controlled release pharmaceutical compositions comprising mesalamine particles, wherein the particles are granulated with hydrophilic polymer, compressed into tablets, and optionally coated with an enteric polymer.

An aspect of the present invention provides controlled release pharmaceutical compositions comprising mesalamine particles, wherein the particles are granulated with a hydrophilic polymer and further embedded in a hydrophobic matrix.

An aspect of the present invention provides controlled release pharmaceutical compositions that release contained mesalamine in a delayed manner, an extended manner, or in a combination of delayed and extended manners.

In an aspect, pharmaceutical compositions of the present invention comprise a therapeutically effective amount of mesalamine or its pharmaceutically acceptable salts, wherein the pharmaceutical compositions are free from a lipophilic matrix, and exhibit desired in vitro release profiles and in vivo performance when orally administered.

In an aspect, compositions of the present invention are free from a lipophilic matrix and contain the active ingredient in an amount of about 75% to about 95% by weight of the compositions.

An aspect of the present invention provides methods of preparing orally administrable pharmaceutical compositions, wherein said compositions are free from a lipophilic matrix, an embodiment comprising:

a. granulating mesalamine particles using a polymer,

b. embedding the granules within a matrix, and

c. coating the matrix with a pH dependent hydrophilic polymer.

An aspect of the present invention provides processes for preparing pharmaceutical compositions wherein mesalamine is granulated using a hydrophobic polymer.

An aspect of the present invention provides processes for preparing pharmaceutical compositions wherein mesalamine is granulated using a hydrophilic polymer.

An aspect of the present invention provides processes for preparing pharmaceutical compositions of the present invention wherein granules are embedded within a hydrophobic matrix.

An aspect of the present invention provides processes for preparing pharmaceutical compositions wherein granules are embedded within a hydrophilic matrix.

An aspect of the present invention provides methods of treating colonic and rectal disorders and the treatment of inflammatory bowel diseases, comprising administering to a patient suffering such disorder an effective amount of a composition of the present invention.

### DETAILED DESCRIPTION

The present invention relates to pharmaceutical compositions comprising mesalamine, including its pharmaceutically acceptable salts, solvates, hydrates, enantiomers, polymorphs, and mixtures thereof, for oral administration.

In the context of the present invention, the terms like “active” or “active agent” or “active substance” or “active pharmaceutical ingredient (API)”, “pharmacologically active agent” “pharmaceutical substance” or “drug” or “drug substance” may be used synonymously for mesalamine.

The term “inflammatory bowel disease” includes, but is not limited to, ulcerative colitis and Crohn's disease. Other diseases contemplated for treatment or prevention by the present invention includes non-ulcerative colitis, and carcinomas, polyps, and/or cysts of the colon and/or rectum. All of these diseases fall within the scope of the term “inflammatory bowel disease” as used in this specification, yet the invention does not require the inclusion of each recited member. Thus, for example, the invention may be directed to the treatment of Crohn's disease, to the exclusion of all the other members; or to ulcerative colitis, to the exclusion of all the other members; or to any single disease or condition, or combination of diseases or conditions, to the exclusion of any other single disease or condition, or combinations of diseases or conditions.

By an “effective” amount or a “therapeutically effective amount” of a drug or pharmacologically active agent is meant a nontoxic but sufficient amount of the drug or agent to provide the desired effect, i.e., relieving the symptoms or lessening the discomfort associated with inflammatory GI tract disorders. It is recognized that the effective amount of a drug or pharmacologically active agent will vary depending on the route of administration, the selected compound, and the species to which the drug or pharmacologically active agent is administered. It is also recognized that one of skill in the art will determine appropriate effective amounts by taking into account such factors as metabolism, bioavailability, and other factors that affect plasma levels of a drug or pharmacologically active agent following administration within the unit dose ranges disclosed further herein for different routes of administration.

The term “controlled release” is intended to refer to any drug-containing formulation in which release of the drug is not immediate, i.e., with a “controlled release” formulation, oral administration does not result in immediate release of the drug into an absorption pool. Controlled release can include delayed release, sustained (or extended) release, and combinations thereof.

An aspect of the present invention relates to orally administrable controlled release pharmaceutical unit dose compositions comprising more than about 0.9 g of mesalamine, wherein said compositions are free from a lipophilic matrix.

An aspect of the present invention relates to orally administrable controlled release pharmaceutical unit dose compositions comprising more than about 0.9 g of mesalamine with pharmaceutically acceptable excipients, wherein said compositions are free from a lipophilic matrix.

In an embodiment of the present invention, a controlled release pharmaceutical composition comprises mesalamine particles, wherein the particles are granulated with a hydrophobic polymer, and further embedded in a hydrophilic matrix,

In another embodiment of the present invention, a controlled release pharmaceutical composition comprises mesalamine particles, wherein the particles are granulated with hydrophilic polymer, compressed into tablets, and optionally coated with an enteric polymer.

In an embodiment of the present invention, a controlled release pharmaceutical composition comprises mesalamine particles, wherein the particles are granulated with a hydrophilic polymer and further embedded in a hydrophobic matrix.

In another embodiment of the present invention, a controlled release pharmaceutical composition comprises mesalamine particles, wherein the particles are granulated with a hydrophobic polymer, compressed into tablets, and optionally coated with an enteric polymer.

In an embodiment, a controlled release pharmaceutical composition of the present invention releases the contained mesalamine in a delayed manner, an extended manner, or a combination of delayed and extended manner.

Bulk density used herein is defined as a ratio of apparent volume to mass of the material taken, called untapped bulk density, and also a ratio of settled volume to mass of material taken, called tapped bulk density. A useful procedure for measuring these bulk densities is described in United States Pharmacopeia 29, Test 616 (Bulk Density and Tapped Density), United States Pharmacopoeial Convention, Inc., Rockville, Md., 2005.

In the present invention, the active pharmaceutical ingredient (“API”) used has untapped bulk density ranging between about 0.15 g/ml to about 0.3 g/ml, and tapped bulk density ranging between about 0.3 g/ml to about 0.5 g/ml.

“Particle size distribution” refers to the distribution of maximum dimensions of particles in a powder. A particle size distribution where 90 volume percent of the particles have sizes less than a specified size is referred to as “D₉₀”. The desired particle size range of API material can be obtained directly from a synthesis process or any known particle size reduction processes can be used, such as but not limited to sifting, milling, micronization, fluid energy milling, ball milling, and the like.

In an embodiment of the present invention, a D₉₀ of mesalamine is less than about 200 μm, or less than about 150 μm.

Carr index, as used herein, refers to compressibility, which is a percentage ratio of the difference between tapped bulk density and untapped bulk density, to the tapped bulk density. Carr index values about 5-15% represent materials with excellent flowability, values about 18-21% represent fair flowability, and values above about 40% represent very poor flowability.

In an embodiment of the invention, the mesalamine used has Carr indexes ranging between about 50% and about 70%.

Generally, the processes to prepare the compositions of the invention include a wet or dry granulation stage that results in the formation of granules. The granules obtained typically have bulk density values ranging between about 0.5 g/ml and about 0.7 g/ml, and tapped bulk density values ranging between about 0.65 g/ml and about 0.85 g/ml. The D₉₀ of such mesalamine granules is less than about 1000 μm, or less than about 850 μm, and the Carr index of these granules typically ranges between about 13% and about 15%.

In certain embodiments of the invention, the API is in the form of a pre-mix with one or more hydrophobic polymers. Such pre-mixes can be prepared by dissolving or dispersing mesalamine with hydrophobic polymer in a suitable solvent system, and then removing the solvent using techniques known in the art, for example spray drying, freeze drying, Buchi Rotavapor drying, and the like.

In the context of the present invention, weight ratios of API to polymer typically range from about 1:0.01 to about 1:10, or from about 1:0.01 to about 1:5, or from about 1:0.01 to about 1:2.5, or from about 1:0.03 to about 1:0.2, in the composition.

A pre-mix of mesalamine with ethylcellulose can be obtained by a process comprising:

A pre-mix of mesalamine with polymers can be obtained by processes comprising:

a. dissolving or dispersing mesalamine in a suitable solvent such as water, an alcohol, a ketone, etc., optionally with at least one pharmaceutically acceptable excipient;

b. adding a polymer to the mixture of a; and

c. removing the solvent by spray-drying the mixture to obtain a powdered pre-mix.

In an embodiment the invention includes processes for preparing premix compositions, wherein at least one pharmaceutically acceptable excipient is an alkalizing agent such as a carbonate or bicarbonate of an alkali or alkaline earth metal.

In an embodiment the invention includes processes to prepare premix compositions wherein an excipient agent is sodium carbonate.

The pre-mix so obtained can further be processed, with pharmaceutically acceptable excipients, to formulate desired dosage forms like tablets and capsules, using techniques known in the art.

Typically, controlled release compositions comprise matrix-type or reservoir-type systems, which can be further coated or granulated to achieve desired in vitro dissolution characteristics.

An orally administrable unit-dose pharmaceutical composition refers to various dosage forms including tablets and capsules. Non-limiting examples of tablets include uncoated tablets, film coated tablets, sugar coated tablets, enteric coated tablets, and the like. Similarly, non-limiting examples of capsules comprise hard gelatin capsules, soft gelatin capsules, and the like, which can be filled with particles, powders, granules, pellets/beads, wafers, films, liquids, viscous semi-solids, and the like.

In one embodiment of the invention, a controlled release pharmaceutical composition comprises mesalamine particles, wherein the particles are granulated with a hydrophobic polymer, pH dependent hydrophilic polymer, or mixtures thereof.

In another embodiment, mesalamine particles are embedded in a hydrophilic matrix comprising hydrophilic polymers, for example: celluloses such as carboxymethyl cellulose sodium, carboxymethyl cellulose, hydroxypropyl methylcellulose or hypromellose (“HPMC”), hydroxy propyl cellulose (HPC), cross-linked sodium carboxymethyl cellulose, cross-linked hydroxypropyl cellulose; carboxymethylamide; potassium methacrylate/divinylbenzene copolymers; polymethylmethacrylates; polyhydroxyalkyl methacrylates; cross-linked polyvinyl pyrrolidones; high-molecular weight polyvinylalcohols; gums such as natural gum, agar, agrose, sodium alginate, carrageenan, fucoidan, furcellaran, laminaran, hypnea, eucheums, gum Arabic, gum ghatti, gum karaya, gum tragacanth and locust bean gum; hydrophilic colloids such as alginates, carbopol and polyacrylamides; other substances such as arbinoglactan, pectin, amylopectin, gelatin, and N-vinyl lactams; polysaccharides; and the like. Combinations of any two or more of these polymers, and other polymers having the required properties are within the scope of the invention.

In a further embodiment of the present invention, a hydrophilic matrix with granulated mesalamine particles embedded therein is further coated with a pH dependent hydrophilic polymer.

Non-limiting examples of hydrophobic polymers include celluloses such as methyl cellulose, ethyl cellulose, cellulose acetates and their derivatives, cellulose acetate phthalate, hydroxypropyl methylcellulose phthalate, cellulose acylate, cellulose diacylate, cellulose triacylate, cellulose acetate, cellulose diacetate, cellulose triacetate, mono-, di- and tri-cellulose alkanylates, mono-, di-, and tri-cellulose arylates, and mono-, di- and tri-cellulose alkenylates, crosslinked vinylpyrrolidones also known as crospovidones, examples of commercially available crospovidone products including but not limited to crosslinked povidone, Kollidon™C [manufactured by BASF Germany)], Polyplasdone™XL, XI-10, and 10 [manufactured by ISP Inc. (USA)], polymethacrylic acid based polymers and copolymers sold under the trade name of EUDRAGIT™ (including Eudragit RL and RS, NE-30D, Eudragit S, Eudragit L), zein, and aliphatic polyesters. Other classes of polymers, copolymers of these polymers or their mixtures in various ratios and proportions as required are within the scope of this invention without limitation. Of course, any other polymers, which demonstrate similar characteristics, are also acceptable in the working of this invention.

The following “lipophilic matrix” materials are among those excluded from the scope of the present invention: unsaturated and/or hydrogenated fatty acids, and salts, esters or amides thereof; fatty acid mono-, di- or tri-glycerides; waxes, ceramides; cholesterol derivatives; and mixtures thereof. These substances typically have melting points ranging between about 40° C. to about 90° C.

In the context of the present invention, weight ratios of mesalamine to total polymers in a composition (including hydrophilic and hydrophobic polymers) typically range from about 1:0.01 to about 1:10, or from about 1:0.01 to about 1:7.5, or from about 1:0.01 to about 1:2.5.

In an embodiment, a weight of orally administrable controlled release pharmaceutical unit dose composition comprising more than about 0.9 g of mesalamine of the present invention ranges between about 1.2 g and about 1.8 g, or between about 1.3 g and about 1.6 g.

One or more pharmaceutically acceptable excipients may optionally be used in the preparation of particles or granulation of particles, and in converting the granules into finished dosage form. These pharmaceutically acceptable excipients include but are not limited to: diluents such as microcrystalline cellulose (MCC), silicified MCC (e.g. Prosolv™ HD 90), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders such as acacia, guar gum, alginic acid, dextrin, maltodextrin, methylcelluloses, ethylcelluloses, hydroxyethyl celluloses, hydroxypropyl celluloses (e.g. KLUCEL®), hydroxypropyl methylcelluloses (e.g. METHOCEL®), carboxymethylcellulose sodium, povidones (various grades of KOLLIDON®, PLASDONE®), starch and the like; disintegrants such as carboxymethyl cellulose sodium (e.g. Ac-Di-Sol®, Primellose®), crospovidones (e.g. Kollidon®, Polyplasdone®), povidone K-30, polacrilin potassium, starch, pregelatinized starch, sodium starch glycolate (e.g. Explotab®) and the like; surfactants including anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, sodium lauryl sulfate (SLS or SDS), cationic surfactants such as cetylpyridinium chloride monohydrate and hexadecyltrimethylammonium bromide, nonionic surfactants such as N-decanoyl-N-methylglucamine, octyl a-D-glucopyranoside, n-Dodecyl b-D-maltoside (DDM), polyoxyethylene sorbitan esters like polysorbates and the like; plasticizers such as triethyl citrate, acetyltributyl citrate, phosphate esters, phthalate esters, amides, mineral oils, fatty acids and esters, diacetylated monoglycerides, glycerin, triacetin or sugars, fatty alcohols, polyethylene glycol, ethers of polyethylene glycol, and fatty alcohols such as cetostearyl alcohol, cetyl alcohol, stearyl alcohol, oleyl alcohol, myristyl alcohol and the like. Solvents that can be used in processing, including layering or coating operations, include but are not limited to, aqueous solvents such as water, organic volatile solvents such as acetaldehyde, acetone, benzene, carbon disulphide, carbon tetrachloride, 1,2 dichloroethane, dichloromethane, N,N-dimethylformamide, 1,4-dioxane, epichlorhydrin, ethyl acetate, ethanol, ethyl ether, ethylene glycol, 2-ethoxyethanol (acetate), formaldehyde, isopropanolol, methanol, methyl n-butyl ketone, methyl ethyl ketone, 2-methoxyethanol (acetate), perchloroethylene, toluene, 1,1,1-trichloroethane, trichloroethylene, and the like, and mixtures thereof.

Pharmaceutical compositions of the present invention may further include other ingredients, such as but not limited to pharmaceutically acceptable glidants, lubricants, opacifiers, colorants and other commonly used excipients.

In an embodiment, pharmaceutical compositions of the present invention can be prepared by a process comprising:

a. granulating mesalamine particles using a hydrophobic polymer;

b. embedding the granules within a hydrophilic matrix; and

c. coating the hydrophilic matrix with a pH dependent hydrophilic polymer.

In another embodiment, pharmaceutical compositions of the present invention can be prepared by a process comprising:

a. granulating mesalamine particles using a hydrophilic polymer;

b. embedding the granules within a hydrophobic matrix; and

c. coating the hydrophobic matrix with a pH dependent hydrophilic polymer.

Granules comprising mesalamine particles can be formed by any process, such as dry granulation, wet granulation, extrusion-spheronization, and the like. In embodiments, the granulation, optionally with pharmaceutically acceptable excipients like diluents or fillers, can be carried out in various apparatus, such as a rapid mixer granulator (RMG) or fluidized bed processor (with top spray technique). The granules/beads/particles may further be coated with one or more other actives, and are compressed into tablets or filled into capsules by techniques known in the art. Alternatively, tablets can be prepared by a direct compression technique.

Pharmaceutical compositions of the present invention comprise a therapeutically effective amount of mesalamine or its pharmaceutically acceptable salts, wherein the pharmaceutical compositions are free from a lipophilic matrix, and exhibit desired in vitro release profiles and in vivo performance when orally administered.

The pharmaceutical compositions disclosed herein comprise a therapeutically effective amount of mesalamine or its pharmaceutically acceptable salts, and can be used for the treatment of inflammation in a distal part of small intestine and colon, inflammatory bowel disease, and other such disease conditions.

The following examples further describe certain specific aspects and embodiments of the invention and demonstrate the practice and advantages thereof. It is to be understood that the examples are given by way of illustration only and are not intended to limit the scope of the invention in any manner.

### EXAMPLES

### Example 1

### Mesalamine 1.2 g Tablet Composition with Hydrophilic Matrix

| Ingredient | Grams |
|---|---|
| Mesalamine | 1200 |
| Microcrystalline cellulose (Avicel ™ PH101)$ | 54 |
| Hydroxypropyl methylcellulose (HPMC K100M CR)@ | 122 |
| Colloidal silicon dioxide (Aerosil ® 200)^{#} | 12 |
| Isopropyl alcohol (IPA)* | 250 |
| Water* | 250 |
| Magnesium stearate | 12 |
| Subtotal | 1400 |

Enteric coating

| | |
|---|---|
| Eudragit S100** | 80 |
| Talc | 8 |
| Triethyl citrate | 8 |
| Isopropyl alcohol (IPA)* | 900 |
| Total | 1484 |

^{#}Supplied by Degussa Corp.
$Supplied by FMC Corp.
**Chemically poly(methacrylic acid, methyl methacrylate) 1:2, manufactured by Rohm GmbH.
@Supplied by Dow Chemical Co.
*Evaporates during processing.

Manufacturing process:

1. Mesalamine, Avicel PH101, HPMC and Aerosil were sifted through a ASTM #20 mesh sieve.

2. The blend obtained from step 1 was granulated using a mixture of water and IPA.

3. The granules were dried at 65±5° C. until loss on drying (LOD) at 105° C. was less than about 3% w/w.

4. The dry granulate was sifted through ASTM #20 mesh sieve.

5. The blend from step 4 was lubricated with magnesium stearate sifted through ASTM #60 mesh sieve.

6. The above obtained blend was compressed into tablets of average weight of about 1400 mg.

7. Eudragit S100 was dissolved in IPA.

8. Talc and tri ethyl citrate were added to the step 7 solution and mixed for about 5 minutes.

9. The tablets obtained from step 6 were coated to produce a weight gain about 6% w/w.

### Example 2

### Mesalamine 1.2 g Tablet Composition with Hydrophobic Matrix

| Ingredient | Grams |
|---|---|
| Mesalamine | 1200 |
| Microcrystalline cellulose | 54 |
| (Avicel PH101) | |
| Ethylcellulose 7cP | 122 |
| Colloidal silicon dioxide | 12 |
| (Aerosil ® 200) | |
| Isopropyl alcohol (IPA)* | 400 |
| Magnesium stearate | 12 |
| Subtotal | 1400 |

Enteric coating

| | |
|---|---|
| Eudragit S100 | 80 |
| Talc | 8 |
| Triethyl citrate | 8 |
| Isopropyl alcohol (IPA)* | 900 |
| Total | 1484 |

*Evaporates during processing.

Manufacturing Process:

1. Mesalamine, Avicel PH101, ethylcellulose 7 cps and Aerosil were sifted through an ASTM #20 mesh sieve.

2. The blend obtained from step 1 was granulated using IPA

3. The granules were dried at 65±5° C. until loss on drying (LOD) at 105° C. was less than about 3% w/w.

4. The dried granulate was sifted through an ASTM #20 mesh sieve.

5. The blend from step 4 was blended with magnesium stearate sifted through ASTM #60 mesh sieve.

6. The above-obtained blend was compressed into tablets to a target weight of about 1400 mg.

7. Eudragit S100 was dissolved in IPA.

8. Talc and tri ethyl citrate were added to the step 7 solution and mixed for about 5 minutes.

9. The tablets obtained from step 6 were coated to produce a weight gain of 6% w/w.

### Example 3

### Mesalamine 1.2 g Tablet Composition with Hydrophobic Matrix Mixed with Hydrophilic Polymer

| Ingredient | Grams |
|---|---|
| Mesalamine | 1200 |
| Microcrystalline cellulose | 54 |
| (Avicel PH101) | |
| Ethylcellulose 7cP | 60 |
| Colloidal silicon dioxide | 12 |
| (Aerosil 200) | |
| Isopropyl alcohol (IPA)* | 400 |
| Hydroxypropyl methylcellulose | 52 |
| (HPMC K100M CR) | |
| Magnesium stearate | 12 |
| Subtotal | 1390 |

Enteric coating

| | |
|---|---|
| Eudragit S100 | 80 |
| Talc | 8 |
| Triethyl citrate | 8 |
| Isopropyl alcohol (IPA)* | 900 |
| Total | 1473 |

*Evaporates during processing.

Manufacturing Process:

1. Mesalamine, Avicel PH101, ethylcellulose and Aerosil were sifted through a ASTM #20 mesh sieve.

2. The blend obtained from step 1 was granulated using IPA.

3. The granules were dried at 65±5° C. until loss on drying (LOD) at 105° C. was less than about 3% w/w.

4. The dry granulate was sifted through ASTM #20 mesh sieve.

5. The blend from step 4 was blended with HPMC K 100M CR and lubricated with magnesium stearate sifted through ASTM #60 mesh sieve.

6. The above-obtained blend was compressed into tablets with average weight of about 1390 mg.

7. Eudragit S100 was dissolved in IPA.

8. Talc and triethyl citrate were added to the step 7 solution and mixed for about 5 minutes.

9. The tablets obtained from step 6 were coated to produce a weight gain about 6% w/w.

### Example 4

### Mesalamine 1.2 g Tablet Composition with Hydrophilic Matrix Mixed with Hydrophobic Polymer

| Ingredient | Grams |
|---|---|
| Mesalamine | 1200 |
| Microcrystalline cellulose | 54 |
| (Avicel PH101) | |
| Hydroxypropyl methylcellulose | 60 |
| (HPMC K100M CR) | |
| Colloidal silicon dioxide (Aerosil | 12 |
| 200) | |
| Isopropyl alcohol (IPA)* | 250 |
| Water* | 250 |
| Ethyl cellulose 7 cP | 52 |
| Magnesium stearate | 12 |
| Subtotal | 1390 |

Enteric coating

| | |
|---|---|
| Eudragit S100 | 80 |
| Talc | 8 |
| Triethyl citrate | 8 |
| Isopropyl alcohol (IPA)* | 900 |
| Total | 1473 |

*Evaporates during processing.

Manufacturing Process:

1. Mesalamine, Avicel PH101, HPMC K100MCR and Aerosil were sifted through a #20 mesh sieve.

2. The blend obtained from step 1 was granulated using a mixture of water and IPA.

3. The granules were dried at 65±5° C. until loss on drying (LOD) at 105° C. was less than about 3% w/w.

4. The dry granulate was passed through a ASTM #20 mesh sieve.

5. The blend from step 4 was mixed with ethylcellulose and lubricated with magnesium stearate sifted through ASTM #60 mesh sieve.

6. The above obtained blend was compressed into tablets with average weight of about 1390 mg.

7. Eudragit S100 was dissolved in IPA.

8. Talc and triethyl citrate were added to the step 7 solution and mixed for about 5 minutes.

9. The tablets obtained from step 6 were coated to produce a weight gain about 6% w/w.

An in vitro dissolution study was conducted according to Test 711 “Dissolution” in United States Pharmacopeia 29, United States Pharmacopoeial Convention, Inc., Rockville, Md., 2005, with the following conditions and results:

Media: 500 ml of 0.1 N HCl for initial 2 hours, followed by 900 ml of phosphate buffer pH 7.5.

Apparatus: USP apparatus Type II, 100 rpm stirring in 0.1N hydrochloric acid and 50 rpm in pH 7.5 phosphate buffer.

| | |
|---|---|
| | Cumulative % |
| Time | Drug Dissolved |

| (hours) | LIALDA ® | EXAMPLE 4 |
|---|---|---|
| 0 | 0 | 0 |
| 1 | 6 | 14 |
| 2 | 30 | 30 |
| 4 | 64 | 60 |
| 8 | 102 | 102 |

### Example 5

### Mesalamine 1.2 g Tablet Composition with Hydrophilic Matrix Mixed with Hydrophobic Polymer

| Ingredient | Grams |
|---|---|
| Mesalamine | 1200 |
| Microcrystalline cellulose (Avicel | 126 |
| PH101) | |
| Hydroxypropyl methylcellulose | 20 |
| (HPMC K100M CR) | |
| Colloidal silicon dioxide (Aerosil | 12 |
| 200) | |
| Isopropyl alcohol (IPA)* | 250 |
| Water* | 250 |
| Ethylcellulose 7 cP | 20 |
| Magnesium stearate | 12 |
| Subtotal | 1390 |

Enteric coating

| | |
|---|---|
| Eudragit S100 | 80 |
| Talc | 8 |
| Triethyl citrate | 8 |
| Isopropyl alcohol (IPA)* | 900 |
| Total | 1473 |

*Evaporates during processing.

Manufacturing Process:

1. Mesalamine, Avicel PH101, HPMC K100MCR and Aerosil were sifted through a ASTM #20 mesh sieve.

2. The blend obtained from step 1 was granulated using a mixture of water and IPA.

3. The granules were dried at 65±5° C. until loss on drying (LOD) at 105° C. was less than about 3% w/w.

4. The dry granulate was sifted through ASTM #20 mesh sieve.

5. The blend from step 4 was mixed with ethylcellulose and lubricated with magnesium stearate sifted through ASTM #60 mesh sieve.

6. The above-obtained blend was compressed into tablets of average weight of about 1390 mg.

7. Eudragit S100 was dissolved in IPA.

8. Talc and triethyl citrate were added to the step 7 solution and mixed for about 5 minutes.

9. The tablets obtained from step 6 were coated to produce a weight gain about 6% w/w.

### Example 6

### Mesalamine 1.2 g tablet composition with hydrophilic matrix mixed with Hydrophobic Polymer, and Enteric Coated with a Mixture of Eudragit L and S

| Ingredient | Grams |
|---|---|
| Mesalamine | 1200 |
| Microcrystalline cellulose | 54 |
| (Avicel PH101) | |
| Hydroxypropyl methylcellulose | 60 |
| (HPMC K100M CR) | |
| Colloidal silicon dioxide | 12 |
| (Aerosil 200) | |
| Isopropyl alcohol (IPA)* | 250 |
| Water* | 250 |
| Ethylcellulose 7 cP. | 52 |
| Magnesium stearate | 12 |
| Subtotal | 1390 |

Enteric coating

| | |
|---|---|
| Eudragit L100 | 48 |
| Eudragit S100 | 32 |
| Talc | 8 |
| Triethyl citrate | 8 |
| Isopropyl alcohol (IPA)* | 900 |
| Total | 1473 |

*Evaporates during processing.

Manufacturing process was similar to that described in Example 5.

First in vitro dissolution study:

Media: 500 ml of 0.1 N HCl for initial 2 hours, followed by 900 ml of phosphate buffer pH 6.5.

Apparatus: USP apparatus Type II, 100 rpm in 0.1N HCl and 50 rpm in pH 6.5 phosphate buffer.

| | |
|---|---|
| | Cumulative % |
| Time | Drug Dissolved |

| (hours) | LIALDA ® | EXAMPLE 6 |
|---|---|---|
| 0 | 0 | 0 |
| 1 | 1 | 1 |
| 2 | 5 | 6 |
| 4 | 34 | 38 |
| 8 | 78 | 82 |

Second in vitro dissolution study:

Media: 500 ml of 0.1 N HCl for initial 2 hours, followed by 900 ml of phosphate buffer pH 6.8.

Apparatus: USP apparatus Type II, 100 rpm in 0.1N HCl and 50 rpm in pH 6.8 phosphate buffer.

| | |
|---|---|
| | Cumulative % |
| Time | Drug Dissolved |

| (hours) | LIALDA ® | EXAMPLE 6 |
|---|---|---|
| 1 | 2 | 3 |
| 2 | 6 | 8 |
| 4 | 44 | 50 |
| 8 | 102 | 101 |

### Example 7

### Mesalamine 1.2 g Tablet Composition Having Dispersion with Hydrophobic Polymer

| Ingredient | Grams |
|---|---|
| Mesalamine | 1200 |
| Microcrystalline cellulose | 40 |
| (Avicel PH102) | |
| Sodium carbonate | 40 |
| Colloidal silicon dioxide | 12 |
| (Aerosil 200) | |
| Surelease^{#} | 240 |
| Water* | 250 |
| Magnesium stearate | 12 |
| Subtotal | 1364 |

Enteric coating

| | |
|---|---|
| Eudragit S100 | 80 |
| Talc | 8 |
| Triethyl citrate | 8 |
| Isopropyl alcohol (IPA)* | 900 |
| Total | 1446 |

^{#}Aqueous dispersion comprising 25% ethylcellulose by weight, supplied by Colorcon, USA. The solvent component evaporates during processing.
*Evaporates during processing.

Manufacturing Process:

1. Sodium carbonate was dissolved in water.

2. Mesalamine was added to the above solution and dissolved.

3. Surelease was added to the solution of step 2 and stirred to form a dispersion.

4. The dispersion of step 3 was spray dried.

5. The spray dried blend was mixed with Avicel PH102 and Aerosil.

6. The blend from step 5 was mixed with magnesium stearate

7. The above obtained blend was compressed into tablets to a unit weight of about 1364 mg.

8. Eudragit S100 was dissolved in IPA.

9. Talc and triethyl citrate were added to the step 7 solution and mixed for about 5 minutes.

10. The tablets obtained from step 6 were coated to produce a weight gain about 6% w/w.

### Examples 8-9

### Mesalamine 1.2 g Tablet Compositions

mg/Tablet

| Ingredient | Example 8 | Example 9 |
|---|---|---|
| Mesalamine | 1200 | 1200 |
| Microcrystalline cellulose | 26 | 26 |
| (RANCEL ™ RQ 101) | | |
| Hydroxypropyl methylcellulose | 21 | 21 |
| (Methocel E 50 LV) | | |
| Sodium carboxymethylcellulose | 31 | 31 |
| (Aqualon 9M8F) | | |
| Colloidal silicon dioxide (Aerosil | 5 | 5 |
| 200) | | |
| Hypromellose (Methocel E 50 LV) | 21 | 21 |
| Water-Isopropyl alcohol (70:30 v/v)* | 600 | 0.15 |
| Sodium carboxymethylcellulose | 10 | 10 |
| (Aqualon ™ 9M8F) | | |
| Crospovidone | 26 | 26 |
| Colloidal silicon dioxide (Aerosil | 15 | 15 |
| 200) | | |
| Magnesium stearate | 10 | 10 |
| Eudragit S100 | 16.58 | 19.05 |
| Eudragit L100 | 49.72 | 44.45 |
| Diacetylated monoglycerides | 9 | — |
| (Myvacet ™ 9-45K) | | |
| Triethyl citrate | — | 13.52 |
| Polyethylene glycol 6000 | 2.09 | 2 |
| Titanium dioxide | 3.53 | 3 |
| Talc | 11.73 | 11.27 |
| Iron oxide red | 2.35 | 2.25 |
| Isopropyl alcohol* | 790 | 0.7 |
| Water* | 40 | 0.03 |
| Opacode ™ Black S-1-8152HV# | qs | — |

*Evaporates during processing.
#Opacode ™ Black S-1-8152HV contains black iron oxide, shellac, soya lecithin and antifoam DC1510, and is supplied by Colorcon.
RANCEL ™ 101 supplied by RanQ Pharmaceuticals & Excipients Pvt. Ltd., Thane, Mahrashtra, India.
Methocel E 50 LV supplied by Dow, Aqualon 9M8F supplied by Aqualon, Eudragit S 100 and Eudragit L 100 supplied by Rohm, Myvacet 9-45K supplied by Kerri.

Manufacturing Process:

1) Mesalamine was sifted through an ASTM #16 mesh sieve, and microcrystalline cellulose and Aerosil 200 were sifted through an ASTM #30 mesh sieve.

2) Step 1 materials were dry mixed for 10 minutes.

3) Methocel E 50 LV was added to a mixture of isopropyl alcohol and water with stirring until a clear solution was formed.

4) Step 2) was granulated using step 3) solution.

5) Step 4) was dried in a fluid bed dryer at an inlet temperature of about 60-65° C. until loss on drying was not more than 1.5% w/w.

6) The dried granules of step 5) were sifted through an ASTM #16 mesh sieve.

7) Retained particles of step 6) were milled using a 1.5 mm screen. The milled granules were sifted through an ASTM #16 mesh sieve and mixed with granules of step 6).

8) Sodium carboxymethylcellulose, Aerosil 200 and crospovidone were sifted through an ASTM #30 mesh sieve and mixed with step 7) material for 10 minutes in a double cone blender.

9) Magnesium stearate was sifted through an ASTM #40 mesh sieve and blended with step 8) for 10 minutes in a double cone blender.

10) The lubricated blend from step 9) was compressed into tablets.

11) Eudragit S 100 and Eudragit L 100 were added to isopropyl alcohol with constant stirring.

12) Polyethylene glycol was dissolved in water and the solution was added to step 11).

13) Diacetylated monoglyceride (for Example 7) or triethyl citrate (for Example 8) was added to step 12) with stirring.

14) Talc, titanium dioxide, and iron oxide red were dispersed in isopropyl alcohol and passed through a colloid mill for about 15 minutes, then the dispersion was added to step 13).

15) The tablets from step 10) were coated with dispersion from step 14).

16) The coated tablets were imprinted with Opacode black.

The coated tablets were analyzed for their in vitro release profiles in 900 ml of pH 7.2 phosphate buffer dissolution medium, using USP Apparatus II at 50 rpm stirring. The results are tabulated below:

| Time | Cumulative % Drug Dissolved | | |
|---|---|---|---|
| (hours) | LIALDA ® | Example 8 | Example 9 |
| 1 | 22 | 22 | 19 |
| 2 | 37 | 49 | 45 |
| 4 | 75 | 87 | 85 |
| 6 | 94 | 95 | 94 |
| 8 | 97 | 98 | 97 |

We claim:

1. A pharmaceutical formulation, comprising particles of mesalamine and at least one solid pharmaceutical excipient, granulated using a hydrophilic or hydrophobic polymer.

2. The pharmaceutical formulation of claim 1, which is free of a lipophilic matrix.

3. The pharmaceutical formulation of claim 1, having a coating comprising a pH dependent polymer.

4. The pharmaceutical formulation of claim 1, wherein granulated particles are combined with a hydrophilic or hydrophobic polymer.

5. The pharmaceutical formulation of claim 4, having a coating comprising a pH dependent polymer.

6. The pharmaceutical formulation of claim 5, wherein a coating comprising a pH dependent polymer further comprises a plasticizer.

7. The pharmaceutical formulation of claim 6 wherein a plasticizer is a water insoluble plasticizer.

8. The pharmaceutical formulation of claim 6 wherein a plasticizer is a water soluble plasticizer.

9. The pharmaceutical formulation of claim 1, wherein a ratio of mesalamine to total polymer is about 1:0.01 to about 1:10.

10. The pharmaceutical formulation of claim 1, wherein a ratio of mesalamine to total polymer is about 1:0.01 to about 1:2.5.

11. A pharmaceutical formulation, comprising particles of mesalamine and at least one solid pharmaceutical excipient that are granulated using a hydrophilic or hydrophobic polymer, formed granules being combined with a hydrophobic polymer, compressed into tablets, and coated with a pH dependent polymer.

12. The pharmaceutical formulation of claim 11, wherein particles are granulated using a hydrophilic polymer.

13. The pharmaceutical formulation of claim 11, wherein particles are granulated using a hydrophobic polymer.

14. The pharmaceutical formulation of claim 11, wherein formed granules are combined with ethylcellulose.

15. A pharmaceutical formulation, comprising particles formed from a solution comprising mesalamine and having dissolved or dispersed therein a hydrophobic polymer.

16. The pharmaceutical formulation of claim 15, wherein the particles are formed by spray-drying.

17. The pharmaceutical formulation of claim 15, wherein the particles are combined with a hydrophilic or hydrophobic polymer.

18. The pharmaceutical formulation of claim 15, having a coating comprising a pH dependent polymer.

19. The pharmaceutical formulation of claim 15, wherein a weight ratio of mesalamine to hydrophobic polymer is about 1:0.01 to about 1:10.

20. The pharmaceutical formulation of claim 15, wherein a weight ratio of mesalamine to hydrophobic polymer is about 1:0.01 to about 1:2.5.
